(19) United States
(12) Patent Application Publication
(10) Pub. No.: US 2021/0298579 A1
(43) Pub. Date: Sep. 30, 2021

(54) OVERTUBE

(71) Applicants: FUJIFILM Corporation, Tokyo (JP); SRJ Corporation, Tochigi (JP)

(72) Inventors: Motohiko MATSUSHITA, Kanagawa (JP); Hironori YAMAMOTO, Tochigi (JP)

(73) Assignees: FUJIFILM Corporation, Tokyo (JP); SRJ Corporation, Tochigi (JP)

(21) Appl. No.: 17/344,966

(22) Filed: Jun. 11, 2021

Related U.S. Application Data
(63) Continuation of application No. PCT/JP2019/047476, filed on Dec. 4, 2019.

(30) Foreign Application Priority Data
Jan. 24, 2019 (JP) 2019-010448

(51) Int. Cl. A61B 1/00

(52) U.S. Cl. CPC A61B 1/00135; A61B 1/00094; A61B 1/00082

(57) ABSTRACT

Provided is an overtube capable of suppressing closing of a ventilation hole provided in an overtube body during endoscopy. An overtube (10) of the present invention includes an overtube body (70) and a balloon (78) that is disposed between a first position (P1) in a direction of a central axis (A) of the overtube body (70) and a second position (P2) positioned on a proximal end (74) side of the overtube body (70) from the first position (P1). The overtube body (70) is provided with a ventilation hole (94) that allows an outer circumferential surface (70A) and an endoscope insertion passage (71) to communicate with each other. The ventilation hole (94) is provided in a ventilation hole-formed region (70D) provided on the proximal end (74) side from the second position (P2). The ventilation hole-formed region (70D) is in a region within a range of 5 mm or more and 100 mm or less from the second position (P2) toward the proximal end (74) side of the overtube body (70).

### CROSS-REFERENCE TO RELATED APPLICATIONS

The present application is a Continuation of PCT International Application No. PCT/JP2019/047476 filed on Dec. 4, 2019 claiming priority under 35 U.S.C § 119(a) to Japanese Patent Application No. 2019-010448 filed on Jan. 24, 2019. Each of the above applications is hereby expressly incorporated by reference, in its entirety, into the present application.

### BACKGROUND OF THE INVENTION

### 1. Field of the Invention

The present invention relates to an overtube that is inserted into a luminal organ in a body together with an insertion part of an endoscope.

### 2. Description of the Related Art

In the related art, a technique of inserting an insertion part of an endoscope into a digestive tract (also referred to as a luminal organ), such as a large intestine and a small intestine, and performing observation, diagnosis, and treatment of an inner wall surface of the digestive tract is performed in the medical field. The digestive tract, such as the large intestine and the small intestine, is bent in a complicated manner. Thus, it is difficult to transmit a force to a distal end of the insertion part simply by pushing the insertion part of the endoscope, and it is difficult to insert the endoscope into a deep portion.

Thus, a so-called double-balloon endoscope device, in which an inflatable and deflatable balloon is provided at each of the insertion part of the endoscope and a distal end portion of the overtube (also referred to as an endoscope insertion auxiliary tool) covered with the insertion part, is known. In the endoscope device, a balloon control device can individually control the inflation and deflation of each balloon by supplying and sucking air into and from each balloon. Accordingly, the insertion part can be inserted into the deep portion of the digestive tract bent in a complicated manner by alternately inserting the insertion part and the overtube while temporarily fixing each balloon to the digestive tract individually at a predetermined timing.

In endoscopy using such an endoscope device, there is a technique of dragging the digestive tract to a hand side as an operator operates the overtube to be pulled to the hand side after the balloon of the overtube is inflated and the balloon is closely attached to the inner wall surface of the digestive tract. In this case, there is a shortcoming in that the pulling operation of the overtube cannot be smoothly performed in a case where a gas (an existing gas in the intestine and a gas (air or a carbon dioxide gas) supplied from the endoscope) accumulated behind (removal direction) the balloon is compressed to increase an internal pressure of the digestive tract.

An overtube intended to solve the shortcoming is disclosed in JP1998-155733A (JP-H10-155733A), JP2005-205182A, JP2009-022443A, JP2009-022444A, and JP2011-188898A.

In the overtube disclosed in each of JP1998-155733A (JP-H10-155733A), JP2005-205182A, JP2009-022443A, and JP2009-022444A, a ventilation hole is provided from a balloon mounting position of an overtube body to a proximal end side of the overtube body in order to exhaust air accumulated in a gap between the overtube body and the intestinal wall to the outside of the body at the time of the pulling operation of the overtube.

In addition, in the overtube disclosed in JP2011-188898A, ventilation holes on a distal end side and the proximal end side that are near a balloon of the overtube body and a communication passage that communicates with the ventilation holes are provided. In the overtube, at the time of the pulling operation of the overtube, it is possible to exhaust air on the proximal end side of the balloon to the distal end side of the balloon via the ventilation hole on the proximal end side, the communication passage, and the ventilation hole on the distal end side.

### SUMMARY OF THE INVENTION

However, depending on a position for disposing the ventilation hole with respect to the overtube body, the following problems occur in some cases.

For example, in a case of operating the overtube to be pulled, the ventilation hole is closed by the inner wall surface of the digestive tract in some cases depending on the position for disposing the ventilation hole. In a case where the overtube is forcibly operated to be pulled in such a state, an opening edge portion of the ventilation hole slides on the inner wall surface of the digestive tract. Thus, a residue adhered to the inner wall surface infiltrates into the overtube body from the ventilation hole, and a relative sliding operation between the endoscope insertion part and the overtube body deteriorates in some cases. As a result, it is difficult to smoothly perform the pulling operation of the overtube.

In addition, in a case of deflating the balloon and operating the overtube to be pushed to the deep portion, when the ventilation hole is closed by the deflated balloon, there is a possibility that the deflated balloon is caught inside the overtube body from the ventilation hole, and is further sandwiched between an inner circumferential surface of the overtube body and an outer circumferential surface of the endoscope insertion part. Thus, it is difficult to smoothly perform the pushing operation of the overtube.

JP1998-155733A (JP-H10-155733A), JP2005-205182A, JP2009-022443A, JP2009-022444A, and JP2011-188898A do not consider the problems described above at all, and there is no description suggesting a unit for solving the problems.

The present invention is devised in view of such circumstances, and an object thereof is to provide an overtube that can suppress closing of the ventilation hole provided in the overtube body during endoscopy.

According to an aspect of the present invention, in order to achieve the object of the present invention, there is provided an overtube comprising an overtube body that has a distal end, a proximal end, and a central axis and in which an endoscope insertion passage is formed between the distal end and the proximal end along the central axis, and a balloon that is provided on an outer circumferential surface of the overtube body, and is disposed between a first position in a direction of the central axis of the overtube body and a second position positioned on a proximal end side of the overtube body from the first position. The overtube body has a ventilation hole-formed region provided on the proximal end side from the second position, and a ventilation hole that allows the outer circumferential surface and the endoscope insertion passage to communicate with each other is provided in the ventilation hole-formed region. The ventilation hole-formed region is in a region within a range of 5 mm or more and 100 mm or less from the second position toward the proximal end side of the overtube body.

According to the aspect of the present invention, it is preferable that the ventilation hole-formed region is in a region within a range of 15 mm or more and 40 mm or less from the second position toward the proximal end side of the overtube body.

According to the aspect of the present invention, it is preferable that a plurality of the ventilation holes are provided in the ventilation hole-formed region.

According to the aspect of the present invention, it is preferable that the overtube body has a liquid supply port through which a liquid is supplied to the endoscope insertion passage, and it is preferable that in a case of being viewed from the proximal end side of the overtube body, a position of the ventilation hole is in a range of less than 180 degrees clockwise about the central axis from a position of the liquid supply port.

According to the aspect of the present invention, it is preferable that in the case of being viewed from the proximal end side of the overtube body, the position of the ventilation hole is in a range of 45 degrees or more and 135 degrees or less clockwise about the central axis from the position of the liquid supply port.

According to the aspect of the present invention, it is preferable that a region of the overtube body on the proximal end side from the ventilation hole-formed region is a ventilation hole-non-formed region.

According to the aspect of the present invention, it is preferable that a gripping part is provided on the proximal end side of the overtube body, and it is preferable that the gripping part has a discharge hole that communicates with an outer circumferential surface of the gripping part and the endoscope insertion passage.

According to the aspect of the present invention, it is preferable that a ventilation film that selectively allows a gas to pass therethrough without allowing a liquid to pass therethrough is provided in the ventilation hole.

With the present invention, it can be prevented that the ventilation hole provided in the overtube body is closed during endoscopy.

### BRIEF DESCRIPTION OF THE DRAWINGS

FIG. 1 is a system configuration view of an endoscope device having an overtube according to a first embodiment.

FIG. 2 is an enlarged perspective view illustrating a distal end portion of an insertion part on which a balloon is mounted.

FIG. 3 is a side view of the overtube.

FIG. 4 is a cross sectional view of the overtube in which the insertion part is inserted in the overtube.

FIG. 5 is a main part explanatory view illustrating an example in which the insertion part and an overtube body are inserted in a large intestine.

FIG. 6 is an explanatory view illustrating an example of an insertion method of inserting the insertion part of an endoscope into a lumen.

FIG. 7 is an enlarged explanatory view illustrating a state immediately before the overtube is operated to be pulled.

FIG. 8 is an enlarged explanatory view illustrating a state where the overtube is operated to be pulled.

FIG. 9 is a cross sectional view of the deflated balloon.

FIG. 10 is a cross sectional view taken along line M-N of FIG. 3.

FIG. 11 is an explanatory view illustrating a state where the overtube is inserted through an anus and is inserted through a mouth.

FIG. 12 is a front view of a porous film provided in a ventilation hole.

FIG. 13 is an explanatory view illustrating a stored state of an overtube according to a second embodiment.

FIG. 14 is an explanatory view illustrating a state where the overtube body is inserted in the lumen.

FIG. 15 is an explanatory view illustrating a form when the overtube body is inserted.

FIG. 16 is an explanatory view illustrating an example of a range of an opposite region of the overtube.

FIG. 17 is an explanatory view illustrating main parts of the overtube.

FIG. 18 is an explanatory view schematically illustrating a situation where an operator and an assistant perform endoscopy.

FIG. 19 is an explanatory view illustrating a main part configuration of the overtube.

FIG. 20 is a cross sectional view of the overtube body in which a discharge path is formed.

### DESCRIPTION OF THE PREFERRED EMBODIMENTS

Hereinafter, an overtube according to preferable embodiments of the present invention will be described in detail with reference to the accompanying drawings.

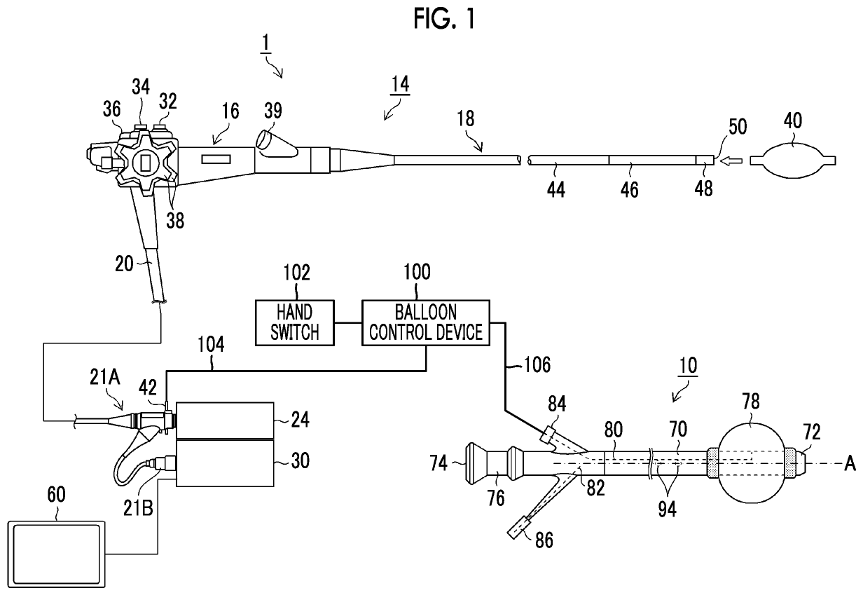

FIG. 1 is a system configuration view of an endoscope device 1 having an overtube 10 according to a first embodiment of the present invention.

The endoscope device 1 illustrated in FIG. 1 comprises an endoscope 14, the overtube 10, and a balloon control device 100. Although an endoscope for a lower digestive tract is given as an example of the endoscope 14, other endoscopes for an upper digestive tract can also be applied.

[Endoscope 14]

The endoscope 14 comprises a hand operation part 16 and an insertion part 18 which is continuously connected to the hand operation part 16. A universal cable 20 is connected to the hand operation part 16. Although not illustrated, the universal cable 20 includes a signal cable, a light guide, and an air supply tube. At a distal end of the universal cable 20, a connector 21A connected to a light source device 24, a connector 21B that is branched from the connector 21A and that is connected to a processor 30 are provided. A monitor 60 is connected to the processor 30.

In addition, in the hand operation part 16, an air supply and water supply button 32, a suction button 34, and a shutter button 36 are arranged to be parallel to each other, and a pair of angle knobs 38 and 38 and a forceps insertion part 39 are provided. Further, the connector 21A is provided with a balloon air supply port 42 for supplying air to a balloon 40 to be described later or for sucking air from the balloon 40. The “air” herein is a gas for inflating the balloon 40 (also including a balloon 78 to be described later), and a type (component) thereof is not particularly limited.

The insertion part 18 has a flexible portion 44, a curved portion 46, and a distal end portion 48 from a proximal end side toward a distal end side of the insertion part 18. The curved portion 46 is remotely curved by moving the pair of angle knobs 38 and 38 provided in the hand operation part 16 rotationally. Accordingly, a distal end surface 50 of the distal end portion 48 can be directed in a desired direction.

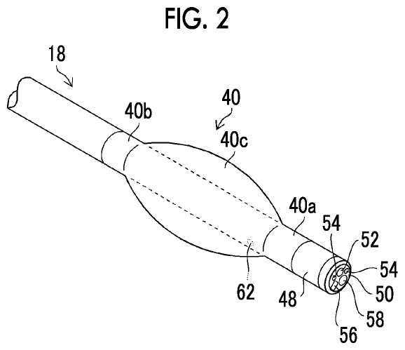

FIG. 2 is an enlarged perspective view illustrating the distal end portion 48 of the insertion part 18.

As illustrated in FIG. 2, the distal end surface 50 of the distal end portion 48 has an observation window 52, a pair of illumination windows 54 and 54, an air supply and water supply nozzle 56, and a forceps port 58. In the distal end portion 48, an imaging element (not illustrated) is provided behind the observation window 52. An observation image is formed on the imaging element and is photoelectrically converted. A signal cable (not illustrated) is connected to the imaging element, and the signal cable is connected to the processor 30 via the insertion part 18, the hand operation part 16, and the universal cable 20, which are illustrated in FIG. 1. Therefore, an electric signal indicating the observation image photoelectrically converted by the imaging element is output to the processor 30, and then is output to the monitor 60 after the signal is appropriately processed in the processor. Accordingly, the observation image is displayed on the monitor 60.

Referring back to FIG. 2, a light emission end of the light guide (not illustrated) is disposed behind each of the pair of illumination windows 54 and 54 in the distal end portion 48. A light incident end of each light guide is connected to the light source device 24 (refer to FIG. 1). Accordingly, an observed part is irradiated with illumination light, which is supplied from the light source device 24 to the light incident end of each light guide, from the light emission end of each light guide via the pair of illumination windows 54 and 54.

An air supply suction port 62 is provided in an outer circumferential surface of the distal end portion 48. The air supply suction port 62 communicates with the balloon air supply port 42 via the air supply tube (not illustrated) inserted from the inside of the insertion part 18 to the connector 21A (refer to FIG. 1). Accordingly, in a case where air is supplied from the balloon air supply port 42, the air is blown out from the air supply suction port 62 to the outside via the air supply tube. In addition, in a case where the air is sucked from the balloon air supply port 42, the air is sucked from the air supply suction port 62 via the air supply tube.

In addition, the balloon 40 formed of various types of elastic bodies is attachably and detachably mounted on the distal end portion 48 of the insertion part 18. The balloon 40 comprises a bulging part 40c at a center thereof and mounting parts 40a and 40b on a distal end side and a proximal end side thereof. In a state where the air supply suction port 62 is disposed on an inner side of the bulging part 40c of the balloon 40, each of the mounting parts 40a and 40b is fixed to the distal end portion 48 through a known method. The bulging part 40c of the balloon 40 configured as described above is inflated into a substantially spherical shape by blowing air from the air supply suction port 62, and the bulging part 40c is deflated by sucking air from the air supply suction port 62.

[Overtube 10]

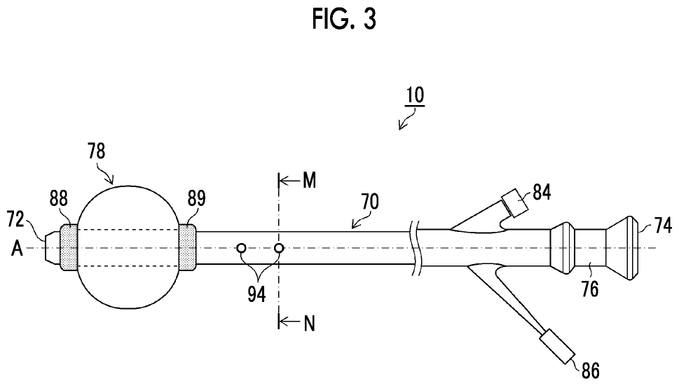

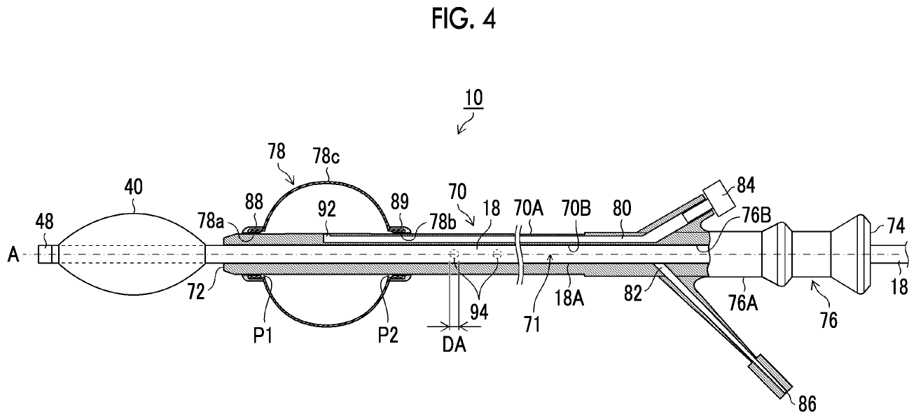

FIG. 3 is a side view of the overtube 10. In addition, FIG. 4 is a cross sectional view of the overtube 10 in a state where the insertion part 18 is inserted in the overtube 10.

As illustrated in FIGS. 3 and 4, the overtube 10 has an overtube body 70. The overtube body 70 is formed of various flexible materials in a tubular shape, has a distal end 72, a proximal end 74, and a central axis A, and an endoscope insertion passage 71 is formed between the distal end 72 and the proximal end 74 along the central axis A. The overtube body 70 has an inner diameter slightly larger than an outer diameter of the insertion part 18. Hereinafter, in the description of each part of the overtube 10, the “distal end side” of each part indicates a side of a direction of the distal end 72, and the “proximal end side” of each part indicates a side of a direction of the proximal end 74.

On the proximal end side thereof, the overtube body 70 comprises a gripping part 76 to be gripped by an operator. The gripping part 76 is formed of various types of hard materials in a tubular shape. For this reason, an outer circumferential surface 70A of the overtube body 70 includes a gripping part outer circumferential surface 76A of the gripping part 76, and an inner circumferential surface 70B of the overtube body 70 includes a gripping part inner circumferential surface 76B. The endoscope insertion passage 71 into which the insertion part 18 is inserted is formed by the inner circumferential surface 70B.

On the other hand, the balloon 78 formed of various types of elastic bodies is mounted on the outer circumferential surface 70A on the distal end side of the overtube body 70. In addition, an air supply and discharge pipe line 80 and a liquid pipe line 82 are formed between the outer circumferential surface 70A and the inner circumferential surface 70B of the overtube body 70.

The air supply and discharge pipe line 80 is formed along the central axis A and is opened as an air supply suction port 92 in the outer circumferential surface 70A, which is positioned on an inner side of the balloon 78. The liquid pipe line 82 is formed to penetrate from the gripping part outer circumferential surface 76A to the gripping part inner circumferential surface 76B. The liquid pipe line 82 is a pipe line for supplying a lubricant such as water between the inner circumferential surface 70B including the gripping part inner circumferential surface 76B and an outer circumferential surface 18A of the insertion part 18.

A balloon air supply port 84 communicating with the air supply and discharge pipe line 80 and a liquid supply port 86 communicating with the liquid pipe line 82 are provided in the gripping part outer circumferential surface 76A.

The balloon air supply port 84 is connected to the balloon control device 100 via a tube 106 (refer to FIG. 1). Therefore, in a case where the balloon control device 100 is driven and air is supplied to the balloon air supply port 84, the air is blown out from the air supply suction port 92 via the air supply and discharge pipe line 80. Accordingly, the balloon 78 is inflated. In addition, in a case where the air is sucked by the balloon control device 100, the air in the balloon 78 is sucked from the air supply suction port 92 via the air supply and discharge pipe line 80. Accordingly, the balloon 78 is deflated.

On the other hand, a lubricant supply unit (not illustrated) such as a syringe is connected to the liquid supply port 86. The liquid supply port 86 hangs downward in a gravity direction due to the weight of the lubricant supply unit, in a state of being connected to the lubricant supply unit. For this reason, the liquid supply port 86 is positioned below the balloon air supply port 84 in the gravity direction in a state where the gripping part 76 is gripped by an operator.

The balloon 78 is provided on the outer circumferential surface 70A of the overtube body 70 in a state of being penetrated by the overtube body 70, and is configured by a bulging part 78c at a center thereof and tubular mounting parts 78a and 78b on the distal end side and the proximal end side of the bulging part 78c. On the outer circumferential surface 70A, the balloon 78 is disposed between a first position P1 of the overtube body 70 in a central axis A direction and a second position P2 positioned on the proximal end side of the overtube body 70 from the first position P1. Herein, for example, the first position P1 is a position where a boundary portion between the bulging part 78c and the mounting part 78a is positioned, and the second position P2 is a position where a boundary portion between the bulging part 78c and the mounting part 78b is positioned. In other words, a distal end of the bulging part 78c is positioned at the first position P1, and a proximal end of the bulging part 78c is positioned at the second position.

A part of the mounting part 78a on the distal end side is folded back to an outer circumferential surface 70A side toward the proximal end side. The mounting part 78a is fixed to the outer circumferential surface 70A of the overtube body 70 by a bonding fixing part 88 made of an adhesive. The bonding fixing part 88 is formed in an annular shape following a circumferential direction of the outer circumferential surface 70A to cover the mounting part 78a and the outer circumferential surface 70A at an edge part thereof.

A part of the mounting part 78b on the proximal end side is folded back to the outer circumferential surface 70A side toward the distal end side. The mounting part 78b is fixed to the outer circumferential surface 70A of the overtube body 70 by an annular bonding fixing part 89 made of an adhesive. The bonding fixing part 89 is formed in an annular shape following the circumferential direction of the outer circumferential surface 70A to cover the mounting part 78b and the outer circumferential surface 70A at an edge part thereof.

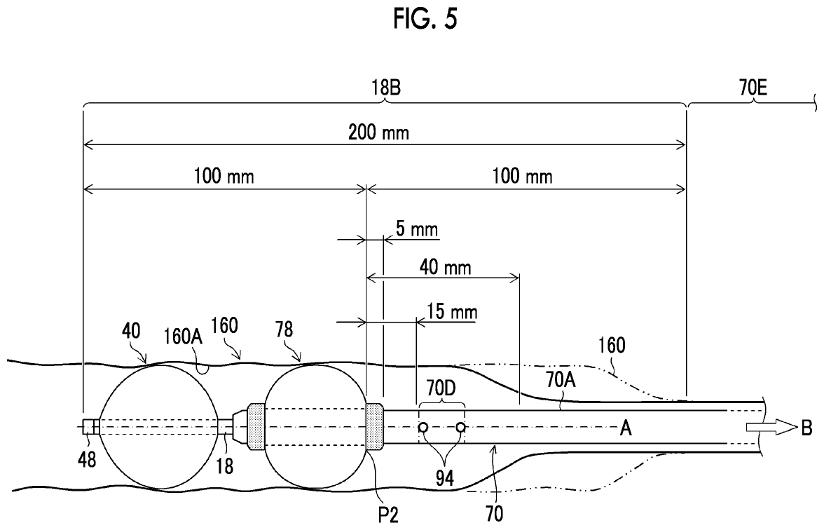

Next, a ventilation hole 94 provided in the overtube body 70 will be described. FIG. 5 is an explanatory view for describing the position of the ventilation hole 94, and illustrates a state where the insertion part 18 and the overtube body 70 are inserted in a large intestine 160, which is the lower digestive tract.

As illustrated in FIG. 5, the overtube body 70 is provided with the two ventilation holes 94 and 94 that allow the outer circumferential surface 70A and the endoscope insertion passage 71 (refer to FIG. 4) to communicate with each other. The ventilation holes 94 and 94 are provided in a ventilation hole-formed region 70D provided on the proximal end side of the overtube body 70 from the second position P2. The ventilation hole-formed region 70D is in a region within a range of 5 mm or more and 100 mm or less from the second position P2 toward the proximal end side of the overtube body 70.

Although the overtube 10 comprising two ventilation holes 94 is given as an example in FIG. 5, the number of ventilation holes 94 may be one or may be three or more. In addition, in a case of providing a plurality of ventilation holes 94, the ventilation holes may be disposed to be spaced apart from each other in the central axis A direction as in FIG. 5, or may be disposed to be spaced apart from each other in the circumferential direction of the outer circumferential surface 70A. In addition, the ventilation holes may be disposed to be spaced apart from each other in the central axis A direction, and may be disposed to be spaced apart from each other in the circumferential direction of the outer circumferential surface 70A.

Herein, the ventilation hole-formed region, which is an element of the embodiment of the present invention, is a region inserted into a body, which is a minimum cylindrical region that includes all of the ventilation holes 94 (which are intended to discharge air accumulated in the digestive tract), among cylindrical regions in the central axis A direction of the overtube body 70. For example, in a case where only one ventilation hole 94 is provided in the overtube body 70, a cylindrical region having a length, which is the diameter of an opening portion of the ventilation hole 94, is the ventilation hole-formed region. In addition, for example, in a case where two ventilation holes 94 and 94 are provided in the overtube body 70 as illustrated in FIG. 5, a minimum cylindrical region (a region indicated by a one-dot chain line in FIG. 5) including the ventilation hole 94 on the distal end side and the ventilation hole 94 on the proximal end side is the ventilation hole-formed region 70D. Further, in a case where three or more ventilation holes 94, 94 . . . are provided in the overtube body 70, a minimum cylindrical region including the ventilation hole 94 on the most distal end side and the ventilation hole 94 on the most proximal end side is the ventilation hole-formed region. A region of the overtube body 70 on the proximal end side from the ventilation hole-formed region is a ventilation hole-non-formed region of the embodiment of the present invention.

[Balloon Control Device 100]

As illustrated in FIG. 1, the balloon control device 100 is connected to the balloon air supply port 42 of the endoscope 14 via a tube 104, is connected to the balloon air supply port 84 of the overtube body 70 via the tube 106, and is also connected to a hand switch 102. The balloon control device 100 supplies air to each of the balloons 40 and 78 or sucks air in each of the balloons 40 and 78 in accordance with a control signal from the hand switch 102. Accordingly, each of the balloons 40 and 78 is individually inflated and deflated.

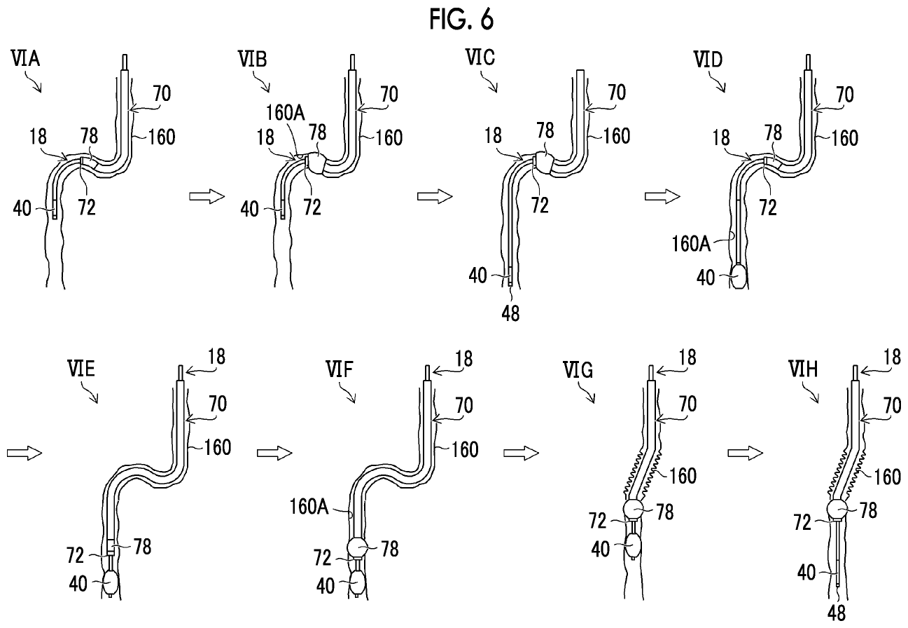

Hereinafter, an example of an insertion method of inserting the insertion part 18 into a deep portion of the large intestine 160 will be described with reference to FIG. 6.

First, as indicated by reference sign VIA of FIG. 6, an operator inserts the insertion part 18 into the large intestine 160 from the anus in a state where the insertion part 18 is covered with the overtube body 70. At this time, the balloon 40 and the balloon 78 are both in a deflated state. Then, the operator inserts the distal end 72 of the overtube body 70 to a bent part of the large intestine 160.

Next, as indicated by reference sign VIB of FIG. 6, air is supplied from the balloon control device 100 (refer to FIG. 1) to the balloon 78 to inflate the balloon 78. Accordingly, the balloon 78 is locked into an inner wall surface 160A of the large intestine 160, and the distal end 72 of the overtube body 70 is fixed to the large intestine 160.

Next, as indicated by reference sign VIC of FIG. 6, the operator inserts only the insertion part 18 of the endoscope 14 into the deep portion of the large intestine 160. Then, as indicated by reference sign VID of FIG. 6, air is supplied from the balloon control device 100 to the balloon 40 to inflate the balloon 40. Accordingly, the balloon 40 is locked into the inner wall surface 160A of the large intestine 160, and the distal end portion 48 of the insertion part 18 is fixed to the large intestine 160.

Next, after the balloon 78 is deflated by sucking air from the balloon 78 by the balloon control device 100 (refer to FIG. 1) as indicated by the reference sign VID of FIG. 6, the operator pushes the overtube body 70 so as to be inserted into the deep portion of the large intestine 160 along the insertion part 18 as indicated by reference sign VIE of FIG. 6. Then, after the distal end 72 of the overtube body 70 is inserted in a vicinity of the balloon 40, air is supplied from the balloon control device 100 (refer to FIG. 1) to the balloon 78 to inflate the balloon 78 as indicated by reference sign VIF of FIG. 6. Accordingly, the balloon 78 is locked into the inner wall surface 160A of the large intestine 160, and the distal end 72 of the overtube body 70 is fixed to the large intestine 160.

Next, as indicated by reference sign VIG of FIG. 6, the operator operates the overtube 10 to be pulled to a hand side. Accordingly, the large intestine 160 is dragged to the hand side and comes into a deflated state.

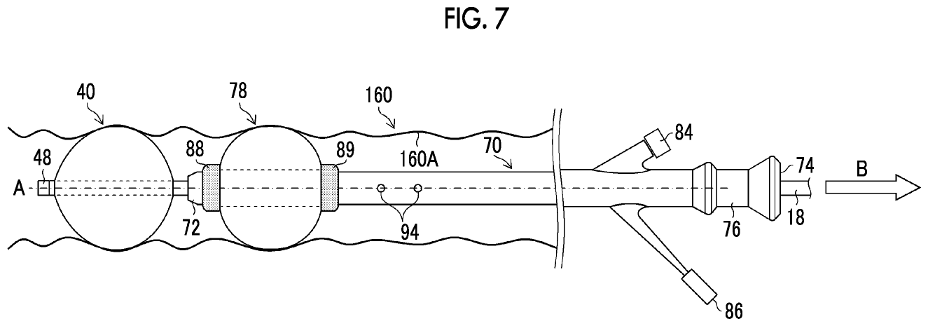

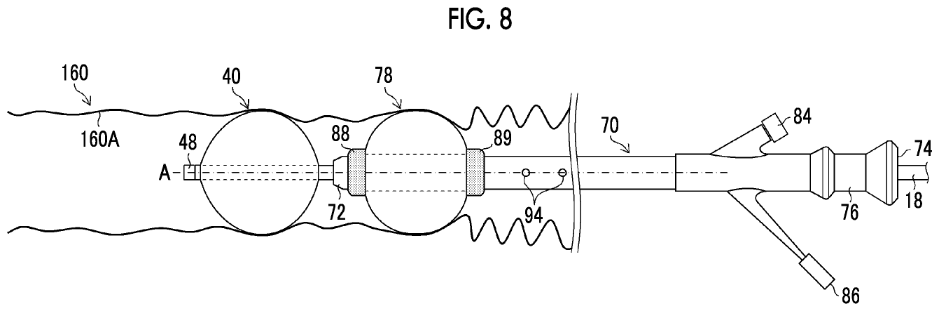

FIG. 7 is an enlarged explanatory view illustrating a state immediately before the operator operates the overtube 10 to be pulled to the hand side, and is a view corresponding to the reference sign VIF of FIG. 6. In addition, FIG. 8 is an enlarged explanatory view illustrating a state where the operator operates the overtube 10 to be pulled to the hand side, and is a view corresponding to the reference sign VIG of FIG. 6.

In a case where the operator operates the overtube body 70 to be pulled to the hand side (an arrow B direction) from the state illustrated in FIG. 7, a gas accumulated in a space formed by the outer circumferential surface 70A, the balloon 78, and the inner wall surface 160A on the proximal end side of the balloon 78 is discharged from the proximal end side of the overtube body 70 to the outside of the body through the ventilation holes 94 and 94 via the endoscope insertion passage 71 (refer to FIG. 5). Accordingly, as illustrated in FIG. 8, the large intestine 160 can be pulled to the hand side without pressing the large intestine 160.

After that, as indicated by reference sign VIH of FIG. 6, air is sucked from the balloon 40 by the balloon control device 100 (refer to FIG. 1) to deflate the balloon 40. Then, the operator further inserts the distal end portion 48 of the insertion part 18 into the deep portion of the large intestine 160. That is, the insertion operation indicated by the reference sign VIC of FIG. 6 is performed again. Accordingly, the distal end portion 48 of the insertion part 18 can be inserted into the deep portion of the large intestine 160. In addition, by repeatedly performing such operation, the distal end portion 48 of the insertion part 18 can be inserted into a deeper portion of the large intestine 160.

Next, the action of the overtube 10 will be described.

As illustrated in FIG. 5, the ventilation hole-formed region 70D where the ventilation holes 94 are provided is in a region within the range of 5 mm or more and 100 mm or less from the second position P2 toward the proximal end side of the overtube body 70. As the ventilation hole-formed region 70D is provided in such a region, the following effects can be obtained.

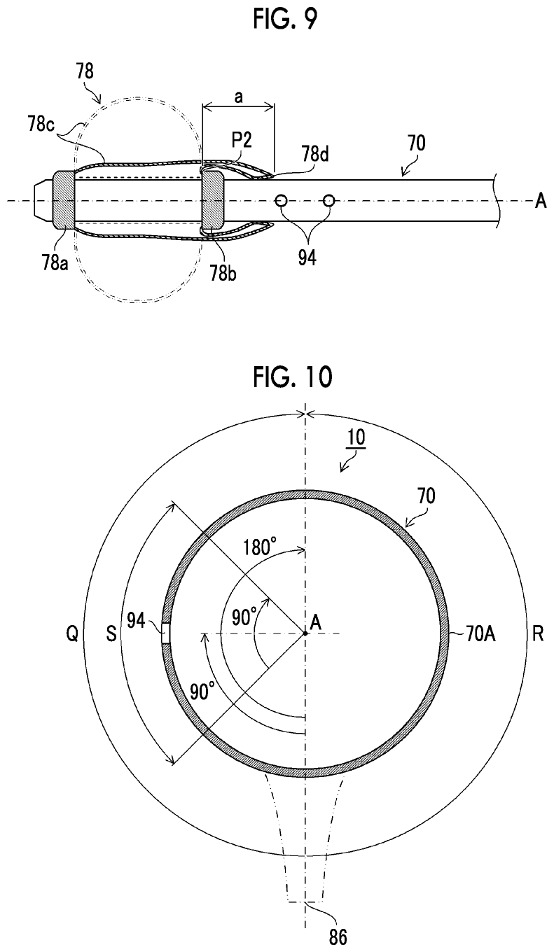

First, the ventilation hole-formed region 70D is in a region of 5 mm or more from the second position P2 toward the proximal end side. For this reason, as indicated by the reference signs VID and VIE of FIG. 6, even in a case where the balloon 78 is deflated and the overtube body 70 is pushed to the deep portion, it is difficult for a deflated proximal end portion 78d of the deflated bulging part 78c to reach the ventilation hole-formed region 70D (refer to FIG. 5) as in the cross sectional view of the balloon 78 illustrated in FIG. 9. Accordingly, it can be prevented that the ventilation holes 94 are closed by the deflated bulging part 78c during endoscopy. A length (a length from the second position P2 toward the deflated proximal end portion 78d) a of the bulging part 78c from the second position P2 toward the proximal end side is slightly different according to the size of the bulging part 78c, but the size of the bulging part 78c, which is set based on the inner diameter of a luminal organ, is generally the same size. Based on the size, by setting the region of 5 mm or more, which is described above, as the ventilation hole-formed region 70D, it can be prevented that the ventilation holes 94 are closed by the deflated bulging part 78c.

In addition, as illustrated in FIG. 5, in a state where the balloons 40 and 78 are inflated, the inner wall surface 160A of the large intestine 160 in a vicinity of the balloons 40 and 78 is expanded in a radial direction due to the inflation of the balloons 40 and 78. For this reason, the inner wall surface 160A in the vicinity of the balloon 78 is a region for which it is difficult to come into contact with the outer circumferential surface 70A of the overtube body 70. However, since it is difficult for a region 70E exceeding 100 mm from the second position P2 toward the proximal end side to be affected by the inflation of the balloon 78, the inner wall surface 160A tends to easily come into contact with the outer circumferential surface 70A. In addition, as another tendency, in a straight region 18B within a range of 200 mm (approximately the length of an ascending colon or a descending colon) from a distal end of the distal end portion 48 of the insertion part 18 immediately before pulling the large intestine 160 to the hand side toward the proximal end side, it tends to be difficult for the inner wall surface 160A to come into contact with the outer circumferential surface 70A since the insertion part 18 and the overtube body 70 are positioned on a straight line. A length from the distal end of the distal end portion 48 of the insertion part 18 to the second position P2 is approximately 100 mm.

Based on the tendency, the ventilation hole-formed region 70D where the ventilation holes 94 are provided is provided in a region of 100 mm or less from the second position P2 toward the proximal end side. Accordingly, it can be prevented that the ventilation holes 94 are closed by the inner wall surface 160A of the large intestine 160 during endoscopy.

As described hereinbefore, in the overtube 10, it can be prevented that the ventilation holes 94 are closed during endoscopy since the ventilation hole-formed region 70D is provided in the region within a range of 5 mm or more and 100 mm or less from the second position P2 toward the proximal end side of the overtube body 70. Accordingly, since it is possible to smoothly perform the pulling operation and pushing operation of the overtube 10, the operability of the overtube 10 by an operator improves.

In the overtube 10, although the ventilation hole-formed region 70D is provided in the region within a range of 5 mm or more and 100 mm or less from the second position P2 toward the proximal end side, the ventilation hole-formed region 70D may be provided, more preferably, in a region within a range of 15 mm or more and 40 mm or less from the second position P2 toward the proximal end side. In this case, the effects can be made more pronounced.

In addition, since the overtube 10 is provided with the plurality of ventilation holes 94, a gas accumulated in the inner wall surface 160A can be efficiently discharged to the outside of the body.

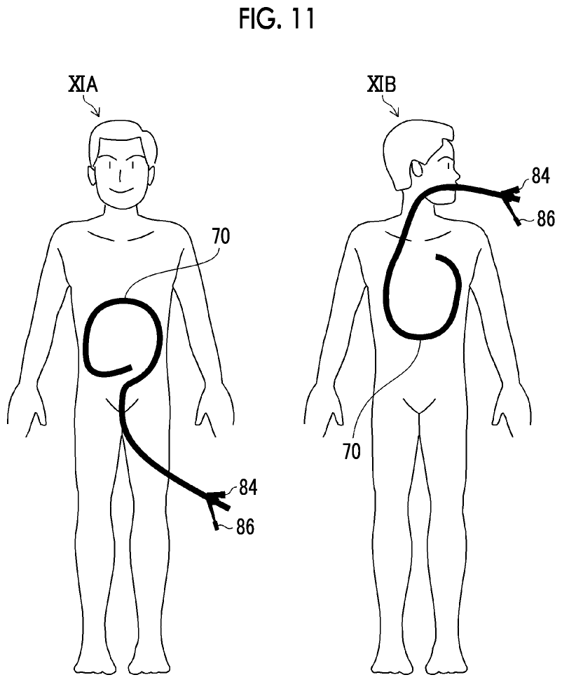

Next, a more preferable position for disposing the ventilation hole 94 will be described with reference to FIGS. 10 and 11.

FIG. 10 is a cross sectional view taken along line M-N of FIG. 3, and illustrates the position of the ventilation hole 94 in a case of being viewed from the proximal end side of the overtube body 70.

As illustrated in FIG. 10, in a case of being viewed from the proximal end side of the overtube body 70, it is preferable that the position of the ventilation hole 94 is within a range Q of less than 180 degrees clockwise from the position of the liquid supply port 86 about the central axis A. In the overtube 10 illustrated in FIG. 10, the position of the ventilation hole 94 is a position of 90 degrees clockwise from the position of the liquid supply port 86 about the central axis A.

Herein, reference sign XIA of FIG. 11 indicates a state where the overtube body 70 is inserted while being deformed in a curved shape along the large intestine (not illustrated) in a case where the overtube body 70 is inserted through the anus in a supine posture. In addition, at this time, since the lubricant supply unit (not illustrated) such as a syringe is connected to the liquid supply port 86, the liquid supply port 86 comes into a state of hanging downward in the gravity direction. In a case where the overtube body 70 is inserted into the large intestine in such a posture, in the outer circumferential surface 70A of the overtube body 70 illustrated in FIG. 10, the outer circumferential surface 70A, which is in a range R of 180 degrees or less counterclockwise from the position of the liquid supply port 86 about the central axis A, tends to easily come into contact with the inner wall surface of the large intestine. Thus, as illustrated in FIG. 10, the overtube 10 is provided with the ventilation hole 94 in the range Q, which is a range for which it is difficult to come into contact with the inner wall surface of the large intestine, in the outer circumferential surface 70A of the overtube body 70. Accordingly, it can be prevented that the ventilation hole 94 is closed by the inner wall surface of the large intestine during endoscopy.

On the other hand, reference sign XIB of FIG. 11 indicates a state where the overtube body 70 is inserted while being deformed in a curved shape along an esophagus (not illustrated) in a case where the overtube body 70 is inserted through the mouth in an abdominal posture as a countermeasure against aspiration pneumonia. In addition, at this time, since the lubricant supply unit (not illustrated) is connected to the liquid supply port 86, the liquid supply port 86 comes into a state of hanging downward in the gravity direction. In a case where the overtube body 70 is inserted into the esophagus in such a posture, the outer circumferential surface 70A in the range R illustrated in FIG. 10 tends to easily come into contact with the inner wall surface of the esophagus. Thus, the overtube 10 is provided with the ventilation hole 94 in the outer circumferential surface 70A in the range Q. Accordingly, it can be prevented that the ventilation hole 94 is closed by the inner wall surface of the esophagus during endoscopy.

As described hereinbefore, in the overtube 10 of the first embodiment, it can be effectively prevented that the ventilation hole 94 is closed during endoscopy even in a case where the overtube body 70 is inserted through the anus and is inserted through the mouth, since the ventilation hole 94 is provided in the range Q.

In addition, although the ventilation hole 94 is provided in the outer circumferential surface 70A in the range Q in the overtube 10 of the first embodiment, the ventilation hole 94 may be provided, more preferably, in a range S of 45 degrees or more and 135 degrees or less clockwise from the position of the liquid supply port 86 about the central axis A in a case of being viewed from the proximal end side of the overtube body 70. In this case, the effects can be made more pronounced.

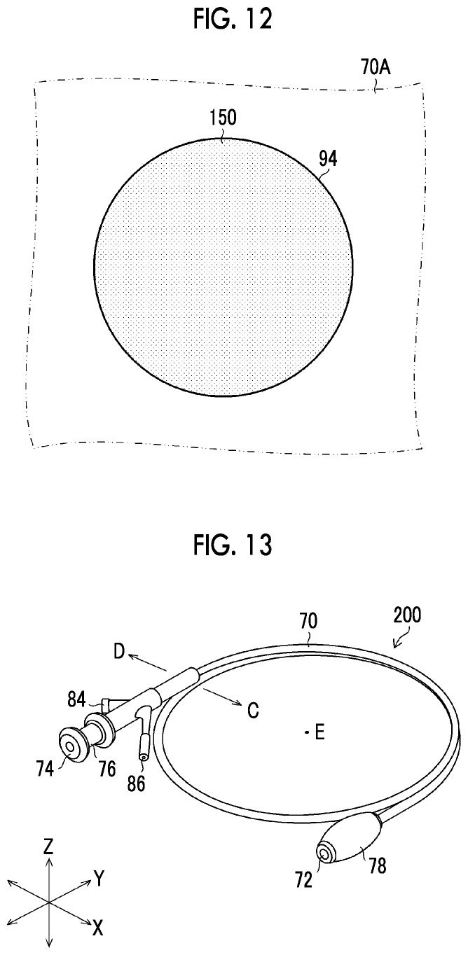

In addition, in the overtube 10 of the first embodiment, it is preferable to provide a porous film 150 illustrated in FIG. 12 in the ventilation hole 94. The porous film 150 is a film that selectively allows a gas to pass therethrough without allowing a liquid to pass therethrough. By providing the porous film 150 in the ventilation hole 94, it can be prevented that a residue included in a bodily fluid infiltrates from the ventilation hole 94 into the endoscope insertion passage 71. Accordingly, a decrease in relative slipperiness between the overtube 10 and the insertion part 18 attributable to the infiltration of the residue can be prevented. The porous film 150 is an example of a ventilation film of the embodiment of the present invention.

In addition, in the overtube 10 of the first embodiment, it is preferable that a diameter DA (refer to FIG. 4) of each ventilation hole 94 is a diameter of 1 mm to 5 mm and that the shape thereof is a circular shape. Since the diameter DA of the ventilation hole 94 is 1 mm or more, it can be prevented that the ventilation hole 94 is clogged by the residue. In addition, since the diameter DA of the ventilation hole 94 is 5 mm or less, a decrease in the strength of the overtube body 70 can be suppressed, and a kink (buckling) of the overtube body 70 can be suppressed. Further, since the ventilation hole 94 has a circular shape, a decrease in the strength of the overtube body 70 can be suppressed, and the kink of the overtube body 70 can be suppressed compared to a ventilation hole having a long hole shape following a circumferential direction of the overtube body 70.

Next, an overtube according to a second embodiment will be described.

FIG. 13 is an external view of an overtube 200 according to the second embodiment. In describing the overtube 200, the same or similar members as those of the overtube 10 of the first embodiment illustrated in FIG. 1 will be assigned with the same reference signs.

The overtube 200 of the second embodiment has the basic configuration of the overtube 10 of the first embodiment, that is, the ventilation hole-formed region 70D is provided in the region within a range of 5 mm or more and 100 mm or less from the second position P2 toward the proximal end side of the overtube body 70. Further, the overtube 200 of the second embodiment has the ventilation hole 94 at a position preferable for disposing in consideration of a curling tendency of the overtube body 70.

As illustrated in FIG. 13, the overtube 200 is stored in a sterilization pack (not illustrated) in a looped state after manufacture.

To describe specifically, the overtube 200 is wound on an X-Y plane of FIG. 13 in a state where the liquid supply port 86 is directed to an inner side of a loop indicated by an arrow C, and the air supply and discharge pipe line 80 is directed to an outer side of the loop indicated by an arrow D. Since the overtube 200 is accommodated in the sterilization pack (not illustrated) in this state, the overtube body 70 has a curling tendency. For this reason, the overtube body 70 of the overtube 200 taken out from the sterilization pack has the curling tendency in a looped direction in the sterilization pack in the natural state as it is.

Herein, to describe with reference to FIG. 6 described above, the insertion part 18 of the endoscope 14 is inserted into the deep portion of the large intestine 160 while bending in a loop along the large intestine 160. For this reason, in a case where the overtube body 70 is inserted into the deep portion of the large intestine 160 along the insertion part 18, the overtube body 70 is also inserted in the same manner while bending in a loop along the loop of the insertion part 18.

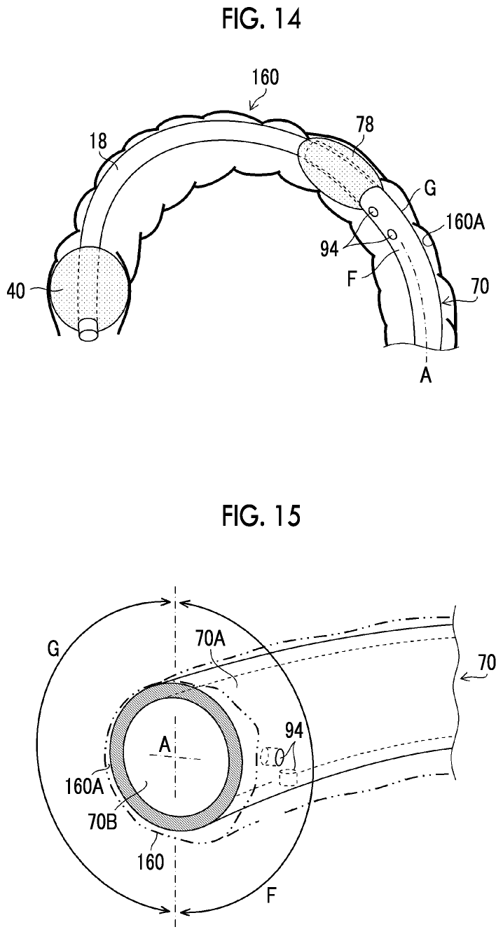

FIG. 14 illustrates a state where the overtube body 70 is inserted in the large intestine 160. In addition, FIG. 15 illustrates a positional relationship between the overtube body 70 inserted in the large intestine 160 and the large intestine 160. As illustrated in FIGS. 14 and 15, since the overtube body 70 has a curling tendency, the overtube body is bent in a loop in the same direction as the curling tendency in the large intestine 160.

As illustrated in FIG. 15, in a case where the overtube body 70 is bent is a loop in the large intestine 160, there are, in the outer circumferential surface 70A, a region F which is bent in a loop on a bending direction side and an opposite region G on an opposite side to the region F due to the curling tendency. The opposite region G of the overtube body 70 inserted in the large intestine 160 tends to easily come into contact with the inner wall surface 160A of the large intestine 160 due to the curling tendency compared to the region F.

The existing ventilation holes 94 are provided in the outer circumferential surface 70A of the overtube body 70. For this reason, in a case where the ventilation holes 94 are provided in the outer circumferential surface 70A in the opposite region G in the ventilation hole-formed region 70D (refer to FIG. 5), it is desirable to ensure that the ventilation holes 94 are not closed by the inner wall surface 160A due to the contact between the opposite region G and the inner wall surface 160A. Thus, in the overtube 200 of the second embodiment, positions for disposing the ventilation holes 94 in the outer circumferential surface 70A are limited to a specific region depending on the curling tendency of the overtube body 70. In other words, a region where the ventilation holes 94 are provided in the ventilation hole-formed region 70D and a region where the ventilation holes 94 are not provided are illustrated in FIG. 5.

Although the ventilation holes 94 are opened, for example, at two places in the outer circumferential surface 70A as illustrated in FIG. 15, the ventilation holes 94 are not formed in the opposite region G of the overtube body 70, and are formed only in the region F on the opposite side to the opposite region G. That is, in a case of the overtube 200 that has a curling tendency, in the ventilation hole-formed region 70D (refer to FIG. 5), the opposite region G is a smooth region where there are no ventilation holes 94, and the ventilation holes 94 are provided only in the specific region F different from the smooth region in the ventilation hole-formed region 70D.

For example, to describe the time when the overtube body 70 is inserted, which is indicated by the reference signs VIC, VID, and VIE of FIG. 6 described above, as an example, the overtube body 70 is inserted in a state where the opposite region G, which is the smooth region, is in contact with the inner wall surface 160A of the large intestine 160, due to the curling tendency thereof as in FIG. 15.

Therefore, in the overtube 200 of the second embodiment, even in a case where the overtube body 70 has a curling tendency, it can be effectively prevented that the ventilation holes 94 are closed by the inner wall surface 160A during endoscopy. Accordingly, the pulling operation of the overtube 200 can be smoothly performed.

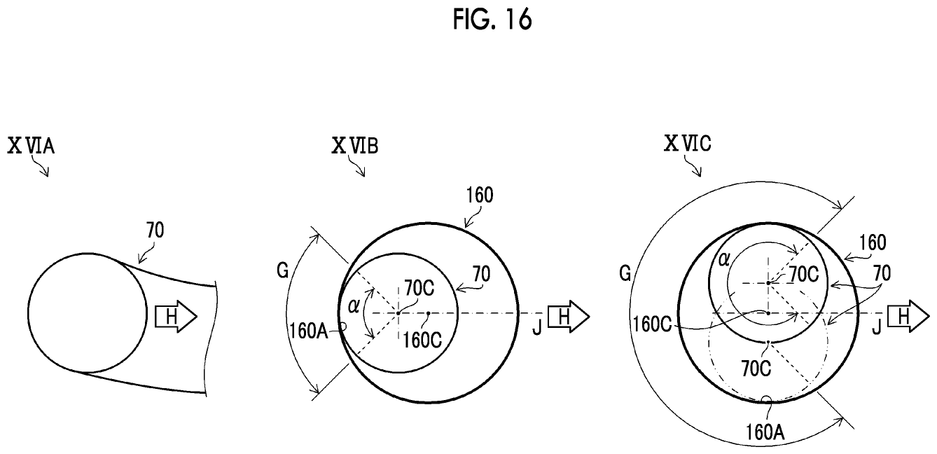

Next, an example of a range of the opposite region G will be described with reference to FIG. 16.

Reference sign XVIA of FIG. 16 is an explanatory view illustrating a curling tendency direction of the overtube body 70, and the curling tendency direction is indicated by an arrow H. The cross sectional view indicated by reference sign XVIB of FIG. 16 illustrates a state where the opposite region G is in contact with the inner wall surface 160A due to the curling tendency of the overtube body 70 indicated by the arrow H. In addition, in the cross sectional view indicated by reference sign XVIC of FIG. 16, a case where the overtube body 70 is displaced upward from the position of the reference sign XVIB is indicated by a solid line, and a case where the overtube body is displaced downward from the position of the reference sign XVIB is indicated by a two-dot chain line. Although the outer diameter of the overtube body 70 is 13.2 mm and the inner diameter of the large intestine 160 is 20 mm in FIG. 16, the dimensions are examples.

A form in which the overtube body 70 is inserted, which is indicated by the reference sign XVIB, is a form in which the overtube body is inserted in a state where a center 70C (the same as the central axis A) of the overtube body 70 and a center 160C of the large intestine 160 are positioned on the same horizontal line (a line parallel to an arrow H direction) J and a state where the opposite region G of the overtube body 70 is in contact with the inner wall surface 160A.

Herein, in a case where the central angle of the overtube body 70 is a, the horizontal line J is 0°, the upper side of the horizontal line J is a + side, and the lower side is a − side, the range of at least α≤±45° is preferably the opposite region G. Accordingly, in a case of a contact form like the reference sign XVIB of FIG. 16, a probability that the opposite region G within the range of at least α≤±45° comes into contact with the inner wall surface 160A is high, which is effective. The central angle a is an angle about the axis about the center 70C.

In addition, it is more preferable to set a range of at least α≤±135° as the opposite region G in consideration of the fact that the overtube body 70 is displaced in an up-and-down direction with respect to the position of the reference sign XVIB. Accordingly, as indicated by the reference sign XVIC of FIG. 16, a probability that the opposite region G within the range of at least α≤±135° comes into contact with the inner wall surface 160A is high, which is effective.

Although the range of α≤±90° is the range of the opposite region G in the overtube 200 illustrated in FIG. 15, the range of the opposite region G is not limited to the range of α≤±90° unlike as described in the examples of the reference signs XVIB and XVIC of FIG. 16, and may be set, for example, depending on the outer diameter of the overtube body 70.

In addition, in the overtube 200, the plurality of (two, in FIG. 14) ventilation holes 94 are provided only in the region F. Accordingly, since a gas accumulated in the space in the inner wall surface 160A can be efficiently discharged to the outside of the body, the large intestine 160 can be smoothly pulled.

In a case of providing the plurality of ventilation holes 94 in the region F, the plurality of (two, in FIG. 14) ventilation holes 94 and 94 may be provided to be spaced apart from each other in the central axis A direction of the overtube body 70 as illustrated in FIG. 14.

In addition, in a case of providing the plurality of ventilation holes 94 in the region F, it is preferable to form at least opening positions of the ventilation holes 94 in the circumferential direction of the outer circumferential surface 70A at positions different from each other as illustrated in FIG. 15. Accordingly, the plurality of ventilation holes 94 can be prevented from simultaneously being closed by the inner wall surface 160A.

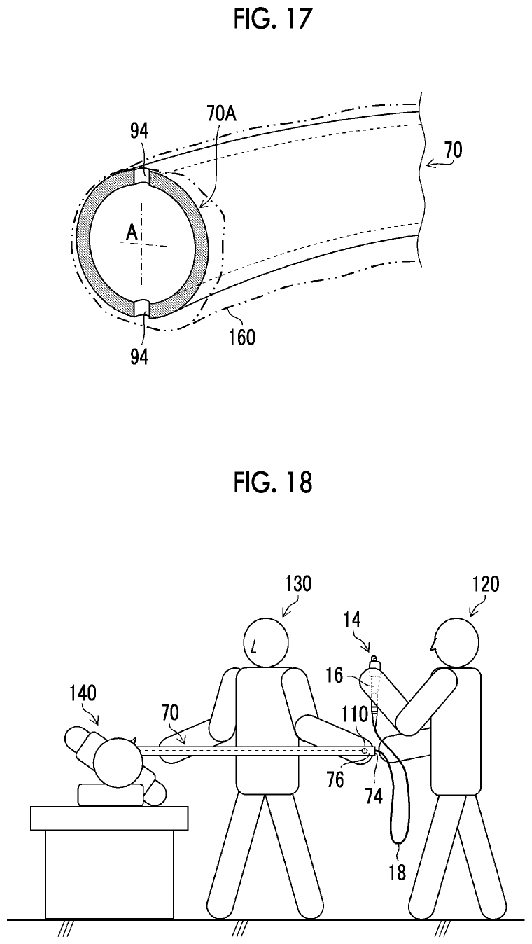

In addition, as illustrated in FIG. 17, the plurality of ventilation holes 94 and 94 may be provided at positions along the same circumferential direction of the overtube body 70. For example, in a case where the two ventilation holes 94 and 94 are provided at an interval of 180 degrees in the same circumferential direction as in FIG. 17, the two ventilation holes 94 and 94 can be simultaneously provided through one punching process.

In addition, in the overtube 200, as in the overtube 10 illustrated in the cross sectional view of FIG. 5, each of the balloon air supply port 84 and the liquid supply port 86 is positioned on an intersecting line in an outer circumferential surface 70A where an imaginary plane (the page of FIG. 5) including the central axis A of the overtube body 70 and the outer circumferential surface 70A intersect each other.

In the overtube 200 configured as described above, since a height in a thickness direction of the overtube 200 when the overtube 200 is looped on the X-Y plane, that is, a Z-direction orthogonal to the X-Y plane, is low as illustrated in FIG. 13, the thickness of the sterilization pack can be made small. The same also applies to the overtube 10 of the first embodiment.

In addition, in the overtube 200, it is more preferable to dispose a liquid supply port 86 side of which a protruding amount from the overtube body 70 is larger than the balloon air supply port 84, on the inner side of the curling tendency, and to dispose the balloon air supply port 84 on the outer side of the curling tendency. Accordingly, since the diameter of the overtube 200 when the overtube 200 is wound is small, the sterilization pack can be miniaturized. The same also applies to the overtube 10 of the first embodiment.

Although the endoscopy illustrated in FIG. 6 is basically performed by one operator, the endoscopy may be performed with the help of an assistant in some cases. Hereinafter, an example in which the operator and the assistant perform endoscopy will be described.

FIG. 18 is an explanatory view schematically illustrating a situation where an operator 120 and an assistant 130 perform endoscopy on a subject 140.

As illustrated in FIG. 18, the operator 120 performs operation of holding the hand operation part 16 of the endoscope 14 with the left hand, holding the insertion part 18 with the right hand, and inserting the insertion part 18 into the large intestine 160 (refer to FIG. 6) of the subject 140 via the overtube body 70. On the other hand, the assistant 130 performs operation of holding the gripping part 76 of the overtube body 70 with the left hand, and holding the distal end side of the overtube body 70 with the right hand to insert or pull the overtube body 70 into or from the large intestine 160 of the subject 140.

In such endoscopy, when the assistant 130 operates the overtube body 70 to be pulled, that is, in a case of pulling the large intestine 160 to the hand side, a liquid (particularly a bodily fluid), which has flowed into the endoscope insertion passage 71 from the ventilation holes 94 of the overtube body 70, is discharged from the proximal end 74 of the overtube body 70 to the outside in some cases.

In such a case, since the operator 120 faces the proximal end 74 of the overtube body 70, it is desirable that the liquid discharged from the proximal end 74 does not adhere to the operator 120.

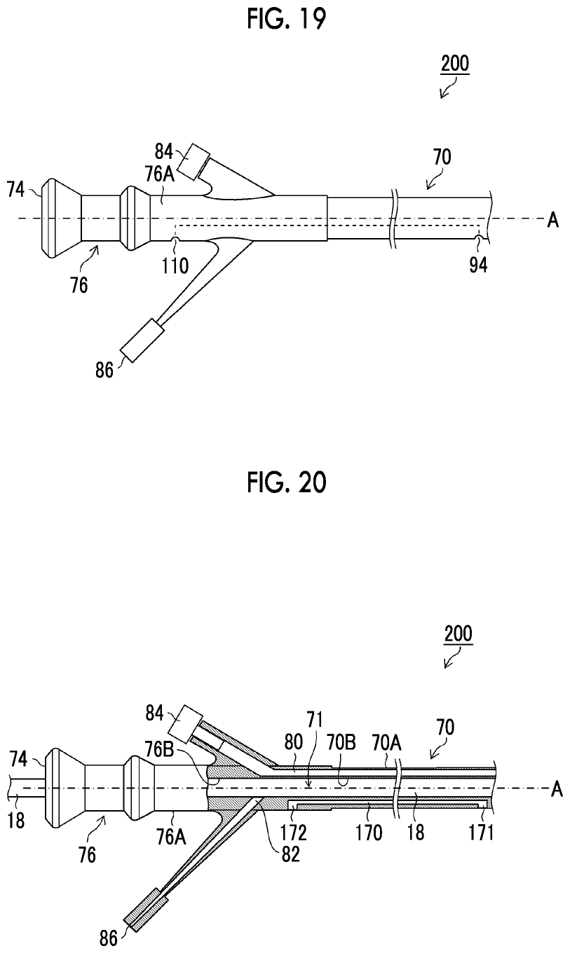

Thus, in the overtube 200, as illustrated in FIG. 19, a gripping part ventilation hole 110 that allows the gripping part outer circumferential surface 76A and the gripping part inner circumferential surface 76B (refer to FIG. 4) to communicate with each other is opened in the gripping part outer circumferential surface 76A of the gripping part 76. The gripping part ventilation hole 110 communicates with the ventilation hole 94 and an opening (not illustrated) of the proximal end 74 via the endoscope insertion passage 71. In other words, the ventilation hole 94 communicates with the gripping part ventilation hole 110 positioned outside the body and the opening of the proximal end 74 via the endoscope insertion passage 71.

In the overtube 200 having the gripping part ventilation hole 110, a liquid flowing into the endoscope insertion passage 71 from the ventilation hole 94 in a case of pulling the large intestine 160 can be discharged to the outside from the gripping part ventilation hole 110 of the gripping part 76. Accordingly, since the amount of liquid discharged from the proximal end 74 of the overtube body 70 is small, the liquid discharged from the proximal end 74 can be prevented from adhering to the operator 120.

It is preferable that a position where the gripping part ventilation hole 110 is formed with respect to the gripping part 76 is formed on a surface of the gripping part outer circumferential surface 76A positioned on a lower side in the gravity direction when the overtube 200 is used. Accordingly, a liquid discharged from the gripping part ventilation hole 110 is discharged downward as it is. In addition, it is preferable that the gripping part ventilation hole 110 is formed at a position on the proximal end side of the overtube body 70 from the liquid pipe line 82 (refer to FIG. 4). Accordingly, the leakage of a lubricant, which is supplied from the liquid supply port 86 to the endoscope insertion passage 71 via the liquid pipe line 82, from the gripping part ventilation hole 110 can be suppressed.

In addition, it is preferable to provide the porous film 150 illustrated in FIG. 12 in the ventilation hole 94 of the overtube 200, and it is preferable to provide the porous film 150 in the gripping part ventilation hole 110 illustrated in FIG. 19. Accordingly, since only a gas is discharged from the gripping part ventilation hole 110 and the proximal end 74, a liquid can be prevented from adhering to the operator 120 and the assistant 130.

In addition, it is preferable to provide the gripping part ventilation hole 110 provided in the overtube 200 of the second embodiment in the overtube 10 of the first embodiment illustrated in FIG. 1. Also in this case, it is preferable to provide the porous film 150 illustrated in FIG. 12 in the gripping part ventilation hole 110.

Although the overtube body 70 of the overtube 200 of the second embodiment comprises, as gas discharge paths of the large intestine 160, a path for discharging a gas from the ventilation hole 94 to the outside of the body via the endoscope insertion passage 71 through the proximal end 74 and a path for discharging the gas from the ventilation hole 94 to the outside of the body via the endoscope insertion passage 71 through the gripping part ventilation hole 110, the discharge path is not limited thereto.

For example, as in the cross sectional view of the overtube body 70 of the overtube 200 of the second embodiment, which is illustrated in FIG. 20, a ventilation passage 170 may be formed along the central axis A, between the outer circumferential surface 70A and the inner circumferential surface 70B of the overtube body 70. The distal end side of the ventilation passage 170 is opened as a ventilation hole 171 at a position corresponding to the ventilation hole 94 described above in the outer circumferential surface 70A of the overtube body 70. In addition, the proximal end side of the ventilation passage 170 is opened as a discharge hole 172 in the gripping part outer circumferential surface 76A on the proximal end side of the overtube body 70 (outside of the body). Therefore, the overtube body 70 of FIG. 20 allows a gas in the large intestine 160 to be discharged from the ventilation hole 171 via the ventilation passage 170 through the discharge hole 172. In addition, it is preferable to provide the porous film 150 illustrated in FIG. 12 in the ventilation hole 171 illustrated in FIG. 20. Accordingly, since only a gas is discharged from the discharge hole 172, a bodily fluid can be prevented from adhering to the operator 120 and the assistant 130. The same also applies to the overtube 10 of the first embodiment.

Although the present invention has been described hereinbefore, the present invention is not limited to the examples, and it is evident that various improvements and modifications may be made without departing from the gist of the present invention. Although the overtube 10 used in the double-balloon endoscope device 1 has been described as an example in the examples, the present invention can also be applied to an overtube used in a single-balloon device.

Although the second embodiment has been described on the premise of having the configuration of the first embodiment, the invention is not limited thereto, and may have a configuration of only the characteristic parts of the second embodiment as another invention. In this case, an effect of the curling tendency of the overtube body 70 can be prevented.

(Appendix)

As understood from the description of the embodiments, the present specification includes disclosure of various technical ideas including the following invention.

(Appendix 1)

An overtube comprising:
an overtube body that has a distal end, a proximal end, and a central axis, allows an insertion part of an endoscope, which is to be inserted into a lumen, to be inserted thereinto, and has a curling tendency;
a balloon that is mounted on an outer circumferential surface of the overtube body; and
at least one or more ventilation holes that are opened in the outer circumferential surface and allow the outer circumferential surface and an inner circumferential surface of the overtube body to communicate with each other,
in which an opposite region in the outer circumferential surface, which is on an opposite side to a region on a bending direction side of the overtube body caused by a curling tendency, is a smooth region, and
a ventilation hole is formed only in a specific region different from the smooth region in the outer circumferential surface.

(Appendix 2)

The overtube according to Appendix 1,
in which a plurality of the ventilation holes are opened in the specific region.

(Appendix 3)

The overtube according to Appendix 2,
in which at least opening positions of the ventilation holes in a circumferential direction of the outer circumferential surface are different from each other.

(Appendix 4)

The overtube according to any one of Appendixes 1 to 3,
in which an air supply and discharge pipe line that allows air to be supplied and discharged into and from an inside of the balloon and a liquid pipe line that allows a liquid to be supplied between the inner circumferential surface and the endoscope are formed between the outer circumferential surface and the inner circumferential surface of the overtube body along the central axis of the overtube body,
a balloon air supply port connected to the air supply and discharge pipe line and a liquid supply port connected to the liquid pipe line are provided in the outer circumferential surface at positions on a proximal end side of the overtube body from the balloon, and
each of the balloon air supply port and the liquid supply port is positioned on an intersecting line in the outer circumferential surface where an imaginary plane including the central axis and the outer circumferential surface intersect each other.

(Appendix 5)

The overtube according to any one of Appendixes 1 to 4,
in which the ventilation hole has a circular shape having a diameter of 1 mm to 5 mm.

(Appendix 6)

The overtube according to any one of Appendixes 1 to 5,
in which there is a gripping part gripped by an operator on a proximal end side of the overtube body,
a gripping part outer circumferential surface of the gripping part is included in the outer circumferential surface, and a gripping part inner circumferential surface of the gripping part is included in the inner circumferential surface, and
the overtube further comprises a gripping part ventilation hole that allows the gripping part inner circumferential surface and the gripping part outer circumferential surface of the gripping part to communicate with each other.

(Appendix 7)

The overtube according to any one of Appendixes 1 to 6,
in which a ventilation film that selectively allows a gas to pass therethrough without allowing a liquid to pass therethrough is provided in the ventilation hole.

Explanation of References

1: endoscope device
10: overtube
14: endoscope
16: hand operation part
18: insertion part
18A: outer circumferential surface
18B: straight region
20: universal cable
21A: connector
21B: connector
24: light source device
30: processor
32: air supply and water supply button
34: suction button
36: shutter button
38: angle knob
39: forceps insertion part
40: balloon
40a: mounting part
40b: mounting part
40c: bulging part
42: balloon air supply port
44: flexible portion
46: curved portion
48: distal end portion
50: distal end surface
52: observation window
54: illumination window
56: air supply and water supply nozzle
58: forceps port
60: monitor
62: air supply suction port
70: overtube body
70A: outer circumferential surface
70B: inner circumferential surface
70C: center
70D: ventilation hole-formed region
70E: region
71: endoscope insertion passage
72: distal end
74: proximal end
76: gripping part
76A: gripping part outer circumferential surface
76B: gripping part inner circumferential surface
78: balloon
78a: mounting part
78b: mounting part
78c: bulging part
78d: deflated proximal end portion
80: air supply and discharge pipe line
82: liquid pipe line
84: balloon air supply port
86: liquid supply port
88: bonding fixing part
89: bonding fixing part
92: air supply suction port
94: ventilation hole
100: balloon control device
102: hand switch
104: tube
106: tube
110: gripping part ventilation hole
120: operator
130: assistant
140: subject
150: porous film
160: large intestine
160A: inner wall surface
160C: center
170: ventilation passage
171: ventilation hole
172: discharge hole
200: overtube
P1: first position
P2: second position
A: central axis
F: region
G: opposite region
J: horizontal line
Q: region
R: range
S: range
DA: diameter
α: central angle

What is claimed is:

1. An overtube comprising:
an overtube body that has a distal end, a proximal end, and a central axis and in which an endoscope insertion passage is formed between the distal end and the proximal end along the central axis; and
a balloon that is provided on an outer circumferential surface of the overtube body, and is disposed between a first position in a direction of the central axis of the overtube body and a second position positioned on a proximal end side of the overtube body from the first position,
wherein the overtube body has a ventilation hole-formed region provided on the proximal end side from the second position, and a ventilation hole that allows the outer circumferential surface and the endoscope insertion passage to communicate with each other is provided in the ventilation hole-formed region, and
the ventilation hole-formed region is in a region within a range of 5 mm or more and 100 mm or less from the second position toward the proximal end side of the overtube body.

2. The overtube according to claim 1,
wherein the ventilation hole-formed region is in a region within a range of 15 mm or more and 40 mm or less from the second position toward the proximal end side of the overtube body.

3. The overtube according to claim 1,
wherein a plurality of the ventilation holes are provided in the ventilation hole-formed region.

4. The overtube according to claim 1,
wherein the overtube body has a liquid supply port through which a liquid is supplied to the endoscope insertion passage, and
in a case of being viewed from the proximal end side of the overtube body, a position of the ventilation hole is in a range of less than 180 degrees clockwise about the central axis from a position of the liquid supply port.

5. The overtube according to claim 4,
wherein in the case of being viewed from the proximal end side of the overtube body, the position of the ventilation hole is in a range of 45 degrees or more and 135 degrees or less clockwise about the central axis from the position of the liquid supply port.

6. The overtube according to claim 1,
wherein a region of the overtube body on the proximal end side from the ventilation hole-formed region is a ventilation hole-nonformed region.

7. The overtube according to claim 1,
wherein a gripping part is provided on the proximal end side of the overtube body, and
the gripping part has a discharge hole that communicates with an outer circumferential surface of the gripping part and the endoscope insertion passage.

8. The overtube according to claim 1,
wherein a ventilation film that selectively allows a gas to pass therethrough without allowing a liquid to pass therethrough is provided in the ventilation hole.
